(19) United States
(12) Patent Application Publication
(10) Pub. No.: US 2015/0086423 A1
(43) Pub. Date: Mar. 26, 2015

(54) STERILIZATION TREATMENT METHOD, FORMULATION FOR STERILIZATION USE, SOLID ICE FOR STERILIZATION USE, METHOD AND DEVICE FOR PRODUCING THE SOLID ICE, AND METHOD FOR PRODUCING LIQUID FOR STERILIZATION USE

(71) Applicant: OSAKA UNIVERSITY, Suita-shi (JP)

(72) Inventors: Katsuhisa KITANO, Suita-shi (JP); Satoshi Ikawa, Izumi-shi (JP)

(73) Assignee: OSAKA UNIVERSITY, Suita-shi (JP)

(21) Appl. No.: 14/523,292

(22) Filed: Oct. 24, 2014

Related U.S. Application Data
(63) Continuation of application No. PCT/JP2013/002877, filed on Apr. 26, 2013.

(30) Foreign Application Priority Data
Apr. 27, 2012 (JP) 2012-103800

(51) Int. Cl. A61L 2/18; F25C 1/00; C01B 15/00

(52) U.S. Cl. CPC A61L 2/18; C01B 15/00; F25C 1/00
USPC 422/28; 423/581; 62/66; 62/340

(57) ABSTRACT

A sterilization treatment method includes producing a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid; freezing the plasma-treated solution to produce solid ice and store the solid ice in a frozen state; thawing the solid ice to the plasma-treated solution in which biocidal activity by the superoxide anion radicals (O₂⁻.) or the precursor of the superoxide anion radicals (O₂⁻.) is held; and applying a sterilization treatment by any one of the following: allowing the plasma-treated solution to have a pH value of 4.8 or lower to apply the resultant plasma-treated solution to an object, and applying the plasma-treated solution to an object of which a pH value is 4.8 or lower.

### CROSS-REFERENCE TO RELATED APPLICATIONS

This application is a continuing application, filed under 35 U.S.C. §111(a), of International Application PCT/JP2013/002877, filed on Apr. 26, 2013, the entire contents of which are incorporated herein by reference.

### FIELD

The present invention relates to a sterilization treatment method using plasma and so on, a formulation for sterilization use, solid ice for sterilization use, a method and device for producing the solid ice, and a method for producing liquid for sterilization use.

### BACKGROUND

Low-temperature plasma generated under a high pressure has conventionally attracted attention in the medical field and other fields. Such plasma is called low-temperature plasma, atmospheric pressure plasma, atmospheric pressure low-temperature plasma, non-equilibrium plasma, Low Frequency (LF) plasma, or the like.

Examples of application of such plasma to medical care include sterilizing and disinfecting medical equipment, pharmaceutical agent, and a living body. For example, since the living body is in moist situations, sterilization in liquid is important. It has been known that an enhanced microbicidal activity by which the time required for sterilization is reduced to approximately one hundredth is obtained by emitting plasma onto a liquid of which a pH value has been lowered (WO2009/041049).

In the odontotherapy field, for example, sterilization has been performed using chemical agents, which unfortunately causes a postoperative infectious disease due to insufficient sterilization. In contrast, sterilization using pH adjustment in a liquid and using plasma PM provides a further sterilizing effect.

Another sterilization method has been proposed in which plasma is generated in a manner not to contact a liquid, active species generated by the plasma are electrophoresed to contact with the liquid, and thereby, the liquid is sterilized (WO2011/027542).

An LF plasma jet which is an example of devices capable of generating such plasma is disclosed in WO2008/072390.

However, in order to use plasma for odontotherapy, a plasma generation device should be installed in a place where medical treatment is provided. Further, a pipe line for introducing different gases should be provided in order to generate plasma and produce active species. According to circumstances, another pipe line is needed to emit gases which are to be generated as a byproduct.

The same is similarly applied to the case of sterilizing and disinfecting medical equipment. To be specific, it is necessary to install a plasma generation device in a clinical site and to secure necessary pipe lines.

### SUMMARY

The present invention has been achieved in light of such an issue, and an object thereof is to perform sterilization using plasma in a place where no plasma generation device is installed.

According to an aspect of the present invention, a sterilization treatment method includes producing a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid; freezing the plasma-treated solution to produce solid ice and store the solid ice in a frozen state; thawing the solid ice to return to the plasma-treated solution in which biocidal activity by the superoxide anion radicals (O₂⁻.) or the precursor of the superoxide anion radicals (O₂⁻.) is held; and applying a sterilization treatment by any one of the following: allowing the plasma-treated solution to have a pH value of 4.8 or lower to apply the resultant plasma-treated solution to an object, and applying the plasma-treated solution to an object of which a pH value is 4.8 or lower.

According to another aspect of the present invention, a method for producing solid ice for sterilization use, the solid ice being thawed to a plasma-treated solution to be used as a liquid for sterilization, the method includes producing a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid; and freezing the plasma-treated solution to produce solid ice, the sold ice, when being thawed, returning to the plasma-treated solution in which biocidal activity by the superoxide anion radicals (O₂⁻.) or the precursor of the superoxide anion radicals (O₂⁻.) is held, and using the sold ice produced as the solid ice for sterilization use.

According to yet another aspect of the present invention, a device for producing solid ice for sterilization use, the solid ice being thawed to a plasma-treated solution to be used as a liquid for sterilization, the device includes a plasma-treated solution production device configured to produce a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid; and a freezing device configured to freeze the plasma-treated solution to produce solid ice, the sold ice, when being thawed, returning to the plasma-treated solution in which biocidal activity by the superoxide anion radicals (O₂⁻.) or the precursor of the superoxide anion radicals (O₂⁻.) is held, and to use the sold ice produced as the solid ice for sterilization use.

According to yet another aspect of the present invention, a method for producing a liquid for sterilization use includes producing a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid; freezing the plasma-treated solution to produce solid ice and store the solid ice in a frozen state; and thawing the solid ice stored in the frozen state to return to the plasma-treated solution in which biocidal activity by the superoxide anion radicals (O₂⁻.) or the precursor of the superoxide anion radicals (O₂⁻.) is held, and allowing the plasma-treated solution to have a pH value of 4.8 or lower to use the resultant plasma-treated solution as the liquid for sterilization use.

According to yet another aspect of the present invention, a formulation for sterilization use includes a plasma-treated solution in which active species generated by plasma are diffused; and a cold insulation material formed integrally with the plasma-treated solution, the cold insulation material being to keep the temperature of the plasma-treated solution at 10° C. or lower.

According to the present invention, it is possible to perform sterilization using plasma in a place where no plasma generation device is installed.

According to the present invention, a plasma-treated solution in which active species have been diffused by plasma has a powerful sterilization effect. The plasma-treated solution becomes harmless immediately after being used for sterilization treatment. The plasma-treated solution has therefore no residual toxicity, and is safe for environment.

### BRIEF DESCRIPTION OF DRAWINGS

FIGS. 1(A) and 1(B) are diagrams depicting a sterilization treatment method according to an embodiment of the present invention;

FIG. 2 is a diagram illustrating how active species are diffused with plasma contacting a liquid;

FIG. 3 is a diagram illustrating how active species are diffused with plasma not contacting a liquid;

FIG. 4 is a diagram showing an example of a plasma generation device;

FIG. 5 is a graph showing the relationship between a lapse of a plasma-treated solution and biocidal activity;

FIG. 6 is a graph showing the relationship between a liquid temperature and a half-life of active species;

FIG. 7 is a graph showing the relationship between a liquid temperature and an ultimate concentration of active species;

FIGS. 8(A)-8(C) are diagrams showing examples of a formulation for sterilization use according to an embodiment of the present invention;

FIGS. 9(A)-9(C) are diagrams showing other examples of a formulation for sterilization use according to an embodiment of the present invention;

FIGS. 10(A)-10(E) are diagrams depicting a method for producing solid ice and a sterilization treatment method according to an embodiment of the present invention;

FIG. 11 is a diagram showing another example of a device for producing solid ice for sterilization use;

FIG. 12 is a diagram showing another example of a device for producing solid ice for sterilization use;

FIG. 13 is a diagram showing another example of a device for producing solid ice for sterilization use;

FIGS. 14(A)-14(G) a diagram showing examples of solid ice for sterilization use according to an embodiment of the present invention;

FIG. 15 is a flowchart illustrating an example as to how solid ice for sterilization use is produced;

FIG. 16 is a flowchart illustrating an example as to how a liquid for sterilization use is produced;

FIG. 17 is a flowchart illustrating an example as to how a sterilization treatment method is carried out;

FIG. 18 is a flowchart illustrating another example as to how a sterilization treatment method is carried out;

FIG. 19 is a table of experimental results for verifying a sterilization effect in a plasma-treated water;

FIG. 20 is a graph showing the residual biocidal activity in a plasma-treated water and a time variation;

FIG. 21 is a graph showing the relationship between a freezing temperature of a plasma-treated water and a sterilization effect; and

FIG. 22 is a graph showing the relationship between a frozen and a lapse of a plasma-treated water and a sterilization effect.

### DESCRIPTION OF EMBODIMENT(S)

A method and device according to the present invention may be implemented in various embodiments described below.

A sterilization treatment method may be implemented as discussed below.

To be specific, active species generated by plasma are brought into contact with a liquid. The active species are diffused in the liquid to produce a plasma-treated solution. The plasma-treated solution is applied to an object; thereby the object is sterilized.

For the sterilization treatment, the temperature of the liquid is kept at 10° C. or lower, the produced plasma-treated solution is preserved at a temperature of 10° C. or lower, and the preserved plasma-treated solution is applied to the object. Alternatively, ice is put into the produced plasma-treated solution to cool the same to a temperature around 0° C. or lower. The plasma-treated solution is preserved in the vicinity of 0° C. or lower, and the preserved plasma-treated solution is applied to the object. Yet alternatively, the produced plasma-treated solution is preserved in the ambient temperature of 0° C. or lower, and the preserved plasma-treated solution is applied to the object.

In short, it is preferable that the temperature of the liquid and the plasma-treated solution is always kept at a low temperature of 10° C. or lower before the liquid and the plasma-treated solution are applied to the object. It is further preferable to keep the temperature of the liquid and the plasma-treated solution at a freezing point temperature of 0° C. or so. It is more preferable to keep the temperature of the produced plasma-treated solution at a low temperature below zero.

In essence, in order to prevent active species diffused in a plasma-treated solution MLp from disappearing, it is necessary to keep the temperature of the plasma-treated solution MLp as low as possible. Further, even during the plasma treatment, some of generated active species disappear. The higher the temperature, the more active species disappear. IL is therefore necessary to keep the temperature of the plasma-treated solution MLp as low as possible.

As a method for keeping the temperature of the liquid and the plasma-treated solution at 0° C. or so, a method is employed of freezing a liquid which has not yet been subjected to plasma treatment to ice. In such a case, the ice is exposed to plasma. While the heat of the plasma thaws a part of the surface of the ice to turn the ice to liquid, plasma treatment is applied to the liquid at the same time. This enables all steps for producing a plasma-treated solution to be performed at a temperature of 0° C. or so.

For production of plasma-treated solution, a liquid of which a pH value has been adjusted to become 4.8 or lower is used. Alternatively, an acid liquid is mixed with the produced plasma-treated solution, and the resultant is adjusted to have a pH value of 4.8 or lower. Then, the plasma-treated solution that has been adjusted to have a pH value of 4.8 or lower in this way is applied to an object.

Alternatively, the produced plasma-treated solution is adjusted to have a pH value of 2 or lower. The resultant plasma-treated solution is preserved, and the preserved plasma-treated solution is applied to an object.

Yet alternatively, the plasma treatment lowers a pH value of the liquid; therefore a pH value of the liquid can be adjusted to become 4.8 or lower through the plasma treatment. In essence, methods for adjusting a pH value include a method by performing the plasma treatment on the liquid and a method by mixing an acid liquid or another drug into the liquid.

Yet alternatively, active species generated by plasma are brought into contact with a liquid, and the active species are diffused in the liquid to produce a plasma-treated solution. The plasma-treated solution is frozen to produce solid ice, and the solid ice is stored in a frozen state in a suitable location or container, e.g., in a freezer car or refrigerator. The solid ice is thawed to return to the plasma-treated solution. The resultant plasma-treated solution is applied to an object to apply a sterilization treatment to the object.

At this time, an acid liquid is mixed with the plasma-treated solution obtained by thawing the solid ice to adjust the mixture to have a pH value of 4.8 or lower. The plasma-treated solution which has been adjusted to have a pH value of 4.8 or lower is applied to the object.

A method for producing solid ice for sterilization use may be implemented as discussed below.

To be specific, active species are diffused in a liquid by plasma to produce a plasma-treated solution. The plasma-treated solution is frozen to produce solid ice. The solid ice is used as solid ice for sterilization use.

After being produced, the plasma-treated solution is frozen quickly to produce solid ice within 3 minutes. The production and rapid-freezing of the plasma-treated solution may be performed in parallel with each other. As described earlier, it is preferable to keep the temperature of the liquid and the plasma-treated solution as low as possible.

A liquid having a pH value of 4.8 or lower is preferably used as a pre-frozen liquid. The solid ice for sterilization use may be a produced solid ice housed in a container. The solid ice for sterilization use may be provided by housing the produced plasma-treated solution in a container, and cooling the container to freeze the plasma-treated solution to produce the solid ice. The solid ice housed in the container may be regarded as the solid ice for sterilization use. The container may be a dropper.

Plasma may be emitted to a liquid in a manner to make contact with the liquid; thereby to diffuse active species in the liquid. Alternatively, plasma may be generated in a gas phase in a manner not to make contact with the liquid. Then, active species generated in the gas phase may be brought into contact with the liquid to be diffused in the liquid.

A method for producing a liquid for sterilization use may be implemented as discussed below.

To be specific, active species generated by plasma are brought into contact with a liquid, and the active species are diffused in the liquid to produce a plasma-treated solution. The plasma-treated solution is frozen to produce solid ice, and the solid ice is stored in a frozen state. The solid ice stored in the frozen state is thawed to return to the plasma-treated solution. The resultant plasma-treated solution is used as a liquid for sterilization use.

As described earlier, in order to produce a liquid for sterilization use, it is preferable to always keep the temperature of the liquid and the plasma-treated solution at a low temperature of 10° C. or lower before the liquid and the plasma-treated solution are applied to the object. It is more preferable to keep the temperature of the liquid and the plasma-treated solution at a temperature of 0° C. or so, or below 0° C.

A liquid having a pH value of 4.8 or lower may be used as a pre-frozen liquid. Alternatively, the plasma-treated solution obtained through thawing may be adjusted to have a pH value of 4.8 or lower.

An apparatus for producing solid ice for sterilization use may be implemented as discussed below.

To be specific, the apparatus is configured of a plasma-treated solution production device for producing a plasma-treated solution by diffusing active species in a liquid by plasma, and a freezing device for freezing the plasma-treated solution to produce solid ice.

The plasma-treated solution production device may be provided with a liquid supply device for dropping a liquid in the vicinity of plasma or dropping a liquid to pass through the plasma. The freezing device may receive the liquid dropped by the liquid supply device to freeze the liquid quickly.

The plasma-treated solution production device may generate plasma in the vicinity of the liquid held in a container or in a manner to make contact with the liquid. The freezing device may be so placed as to refrigerate the container. The generation of the plasma-treated solution and the freezing thereof may be performed in parallel with each other.

A device may be provided which houses the generated plasma-treated solution in the container. In such a case, the freezing device cools the container to freeze the plasma-treated solution, so that solid ice is produced. Further, a pH adjustment device may be provided which adjusts a pH value of the plasma-treated solution to become 4.8 or lower.

The plasma-treated solution production device is provided with a plasma generation device for generating plasma. The plasma generation device is provided with a gas supply pipe, an electrode provided in the vicinity of an outlet of the gas supply pipe, and a voltage applying device for applying a high voltage to the electrode.

In order to keep the liquid and the plasma-treated solution which is being generated at low temperature, the freezing device may be used to cool the liquid and the plasma-treated solution. Alternatively, a separate cooling device may be provided.

A formulation for sterilization use may be implemented as discussed below.

To be specific, the formulation for sterilization use includes a plasma-treated solution in which active species generated by plasma are diffused, and a cold insulation material formed integrally with the plasma-treated solution. The cold insulation material is to keep the temperature of the plasma-treated solution at 10° C. or lower.

Preferably, the plasma-treated solution is adjusted to have a pH value of 4.8 or lower. Alternatively, the plasma-treated solution is adjusted to have a pH value of 2.0 or lower. An example of the cold insulation material is ice. Another example of the cold insulation material is a frozen refrigerant. The plasma-treated solution may be housed in a dropper functioning as the container.

As discussed earlier, in order to produce the plasma-treated solution in such a case, it is preferable to maintain the temperature of the liquid and the plasma-treated solution as low as possible.

[First Embodiment of Sterilization Treatment Method]

An embodiment of the sterilization treatment method will be described.

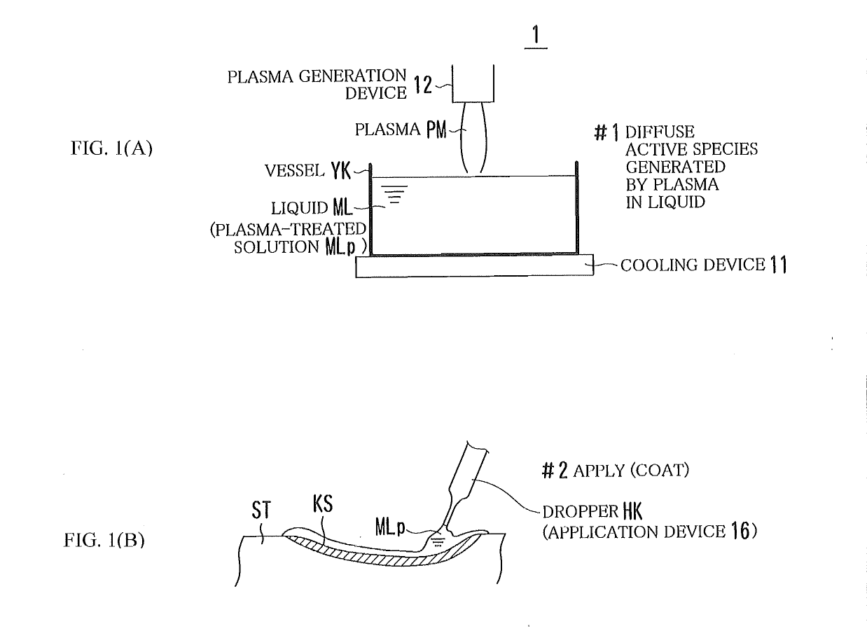

FIG. 1 shows a sterilization treatment method using a plasma-treated solution production device 1.

As illustrated in FIG. 1(A), preparation is made by feeding a vessel YK with a liquid ML. Used as the liquid ML is water like tap water, pure water, physiological saline, an aqueous solution, or various other types of liquid. The liquid ML may be ultrapure water having an electric resistance of 18.2MΩ/Cm or more.

In order to enhance a sterilization effect, it is necessary to adjust the liquid ML to have a pH value of 4.8 or lower before the liquid ML is applied to an object. In this embodiment, as the liquid ML, a liquid that has been adjusted to have a pH value of 4.8 or lower is used. The pH value of the liquid ML may be adjusted to become 4.5 or lower, more preferably to become 3.5 or lower. It is preferable that the pH value be adjusted to become 1 or higher, more preferably 2 or higher to lessen the influence on human body and make the post-processing of the liquid ML easier.

In order to adjust the pH value as described above, there are provided a method of charging acid, or salt that indicates acidity into the liquid ML, a method of blowing a carbonic acid gas into the liquid ML, and so on. For example, citric acid used in food is preferably charged into the liquid ML. In this way, when the pH value is adjusted to make the liquid ML acidic, protons (hydrogen ions) H⁺ in the liquid ML increase.

In order to adjust the pH value in the foregoing manner, a pH adjustment device having an appropriate structure may be used.

The temperature of the liquid ML is kept at a low temperature of 10° C. or lower. The temperature of the produced plasma-treated solution MLp is also kept at a low temperature of 10° C. or lower. To this end, a cooling device 11 may be used. The cooling device 11 may be a device having ice or an appropriate cooling mechanism. Adding ice to the liquid ML works as the cooling device 11. Alternatively, a freezing device 13 described later may be used also as the cooling device 11.

Another arrangement is possible in which the liquid ML itself is frozen to ice. In such a case, the ice is exposed to plasma PM. A part of the ice melts with heat of the plasma PM into the liquid ML, and at the same time, active species are diffused in the liquid ML as described below.

The plasma generation device 12 is used to generate plasma PM, and active species generated by the plasma PM are brought into contact with the liquid ML to be diffused in the liquid ML (Step #1 of FIG. 1(A)). The liquid ML in which the active species generated by the plasma PM are diffused in this way is sometimes referred to as a “plasma-treated solution” or “plasma-treated water”.

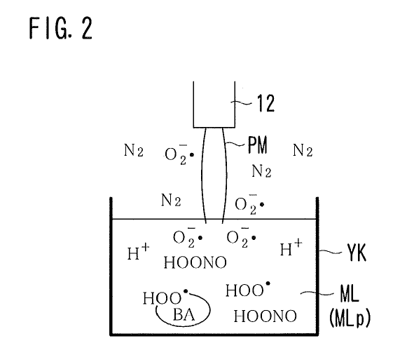

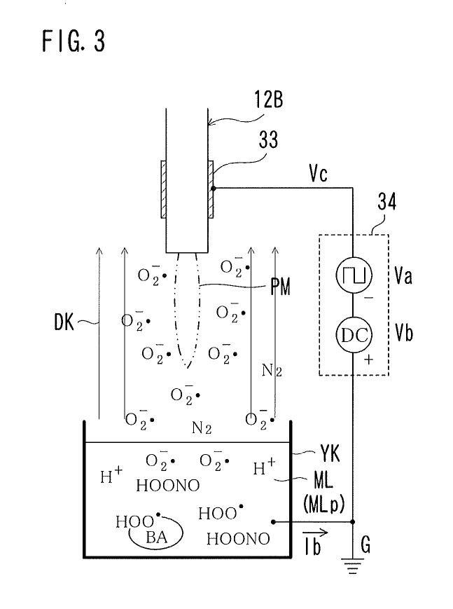

There are two methods to bring the active species into contact with the liquid ML. One is a method in which, as shown in FIG. 2, the plasma PM is emitted to the liquid ML in a manner to make contact therewith. The other is a method in which, as shown in FIG. 3, the active species are electrophoresed without making the plasma PM contact with the liquid ML.

Referring to FIG. 2, the plasma generation device 12 generates plasma PM with the plasma PM making contact with the surface of the liquid ML.

The contact of the plasma PM with the liquid ML causes the active species generated by the plasma PM, in particular, superoxide anion radicals (O₂⁻.) and the derivative (or precursor) thereof to be diffused in the liquid ML, so that the liquid ML turns into a plasma-treated solution MLp.

The microbicidal activity of the plasma-treated solution MLp is associated with acid dissociation equilibrium of the superoxide anion radicals. To be specific, the superoxide anion radicals and the derivative (or precursor) thereof exist in the plasma-treated solution MLp. The derivative generates the superoxide anion radicals gradually, so that the biocidal activity is held.

Referring to FIG. 3, the plasma generation device 12 generates plasma PM with the plasma PM not making contact with the surface of the liquid ML. A voltage applying device 34 applies a voltage Vc containing an AC voltage component Va with a predetermined frequency and a DC bias voltage component Vb with respect to the ground G to an electrode 33 provided in the outer surface of a jet port of a gas supply pipe. The DC bias voltage component Vb has a voltage value of approximately −1 to −10 kV.

The active species such as superoxide anion radicals generated by the plasma PM, and the derivative (or precursor) thereof are electrophoresed by an electric field of the DC bias voltage component Vb, make a contact with the surface of the liquid ML, and are diffused in the liquid ML

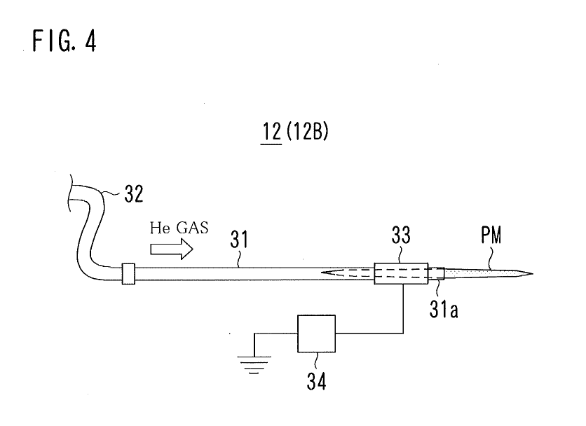

FIG. 4 shows an example of the structure of the plasma generation device 12.

Referring to FIG. 4, the plasma generation device 12 is configured of a gas supply pipe 31, the electrode 33 provided in the vicinity of a jet port 31a of the gas supply pipe 31, the voltage applying device 34, and so on. An AC voltage having a high voltage is applied to the electrode 33, and helium gas is supplied from the gas supply pipe 31. Thereby, plasma PM which is Low Frequency Plasma (LF plasma) is generated in the vicinity of the jet port 31a.

For example, the plasma PM jetted out from the jet port 31a is surrounded by a suitable chamber. Air is introduced into the chamber from the outside to mix with the plasma PM in the chamber, and one end of an appropriate tube is connected to the chamber. A mixed gas (afterglow gas) containing the plasma PM and the air is jetted out from the other end of the tube. In this way, the tube can be used to extend the jet port 31a by a predetermined distance (4 meters, for example), and the mixed gas jetted out from the other end of the tube can be used as the source of radicals similar to the plasma PM.

Aside from the plasma generation device 12, different types of plasma generation devices may be used. For example, a device can be used which converts air into plasma through corona discharge.

Referring back to FIG. 1(B), the produced plasma-treated solution MLp is applied to an object such as an affected area KS of a living body ST (Step #2). To be specific, the plasma-treated solution MLp is put into a dropper HK. The dropper HK is pressed to drop the plasma-treated solution MLp from the bulb of the dropper HK to apply the plasma-treated solution MLp to the affected area KS. In this way, the affected area KS is sterilized and disinfected.

Different types of known droppers may be used as the dropper (spuit, syringe) HK. For example, the dropper HK may be one having a tubular end, a body for holding liquid therein, and an elastic expansion portion. The entirety of the dropper HK may be made of a soft synthetic resin or synthetic rubber. The dropper HK may be so formed that the body and the expansion portion are detachable from each other. If such a dropper having a detachable structure is used, even only a part of the dropper, e.g., even only the body, may be regarded as the “dropper” herein. The dropper HK draws the liquid thereinto or squirts out the liquid by squeezing the expansion portion with fingers or releasing the fingers from the expansion portion. The dropper HK is one example of an application device 16.

The application device 16 may be a robot which is provided with a grip portion and a multi-joint manipulator, and is controlled automatically or controlled by programs. In such a case, for example, the grip portion holds the dropper HK and so on. The manipulator is so controlled and driven to move the dropper HK to the position of the object such as the affected area KS, and then, to drop the plasma-treated solution MLp from the bulb of the dropper HK to the affected area KS.

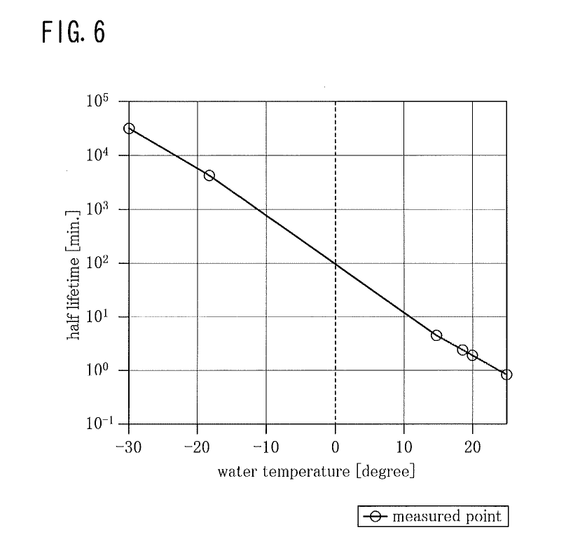

The plasma-treated solution MLp has a powerful sterilization effect. The plasma-treated solution MLp becomes harmless as time passes after being used for sterilization treatment. The plasma-treated solution MLp has therefore no residual toxicity, and is safe for environment. Thus, the plasma-treated solution MLp can be used safely for sterilization treatment on medical equipment, for example. Further, an Arrhenius plot of FIG. 6 shows that, at a temperature of 37° C. corresponding to a body temperature, the active species have a half-life of 4 seconds, which means that the active species are inactivated in a short Lime. The plasma-treated solution MLp has no residual toxicity, and has no harmful influence on the living body. The plasma-treated solution MLp, can therefore be used safely for treatment on living body.

The plasma-treated solution MLp is applicable Lo an object in different ways depending on the situation. The plasma-treated solution MLp may be applied to the object by using a brush or may be sprayed onto the object.

Before the application, the plasma-treated solution MLp may be mixed with another agent or sterilization agent. Alternatively, before the application, another agent or sterilization agent may be dissolved into the plasma-treated solution MLp.

As described earlier, the plasma-treated solution MLp in which active species are diffused has biocidal activity. The biocidal activity of the plasma-treated solution MLp is, however, gradually lost as time advances. As the experiments proceed, it is found that, in order to prolong the biocidal activity of the plasma-treated solution MLp, lowering the temperature of the plasma-treated solution MLp is important.

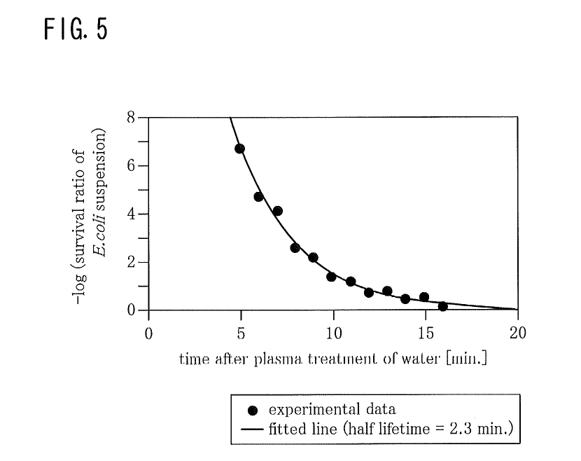

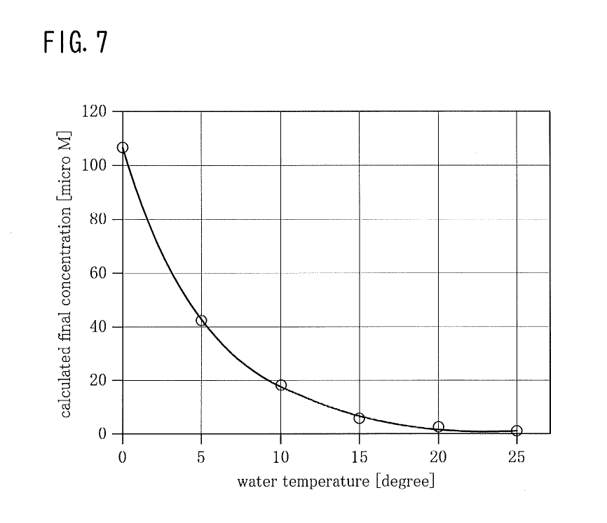

To be specific, FIG. 5 shows the relationship between a lapse of the plasma-treated solution MLp and the biocidal activity; FIG. 6 shows the relationship between a temperature of the plasma-treated solution MLp and a half-life of the active species; and FIG. 7 shows the relationship between a temperature of the plasma-treated solution MLp and an ultimate concentration of the active species.

As shown in FIG. 5, the biocidal activity decreases exponentially with time since the production of the plasma-treated solution MLp. FIG. 6 shows the temperature dependence of the half-life for sterilization characteristics obtained by calculating a half-life of the biocidal activity species based on the time variation in the biocidal activity shown in FIG. 5 and adjusting temperatures of the individual experimental systems.

As indicated in FIG. 6, when the temperature [° C.] of the plasma-treated solution MLp is 25, 20, 19, 15, −18, and −30, the half-life [min.] of active species is 0.8, 1.91, 2.23, 4.25, 4176, and 30240, respectively.

FIG. 6 shows that the half-life for the biocidal activity increases approximately linearly by reducing the temperature of the plasma-treated solution MLp. This reflects the Arrhenius equation in which a logarithm of reaction rate is proportional to a temperature.

As known from FIG. 6, when the temperature is lowered to 10° C., the half-life reaches approximately 10 minutes. Consequently, a practical time is secured for the case where sterilization treatment using the plasma-treated solution MLp is carried out.

Lowering the temperature of the plasma-treated solution MLp increases the half-life of the active species. Therefore, even if a constant amount of active species are supplied by plasma per unit time, a final reached concentration changes. To be specific, for a short half-life, the active species disappear rapidly while being supplied. Accordingly, it is difficult to observe the increase in concentration. FIG. 7 shows the calculation result of dependence of ultimate concentration of active species on temperature.

In FIG. 7, the supply amount of w of active species per unit time is set at 1 μM/min. If the temperature (° C.) of the plasma-treated solution MLp is 25, 20, 15, 10, 5, and 0, the final reached concentration (μM) of active species are 1.2, 2.8, 6.3, 18.8, 43.1, and 107, respectively.

As shown in FIG. 7, the ultimate concentration increases exponentially as the temperature drops. Therefore, the plasma-treated solution MLp having high concentration of active species is obtained by reducing the temperature of the plasma-treated solution MLp or the initial liquid ML.

As known from the above, the time from when the plasma-treated solution MLp is generated until when the plasma-treated solution MLp is applied to the object is preferably as short as possible. For example, in a room temperature of approximately 25° C., it is possible to carry out sufficient sterilization treatment by applying the plasma-treated solution MLp to an object within 1 minute or so from the generation of the plasma-treated solution MLp.

When the temperature of the liquid ML or the plasma-treated solution MLp is set at 10° C. or lower, it is possible to secure the time necessary to apply the plasma-treated solution MLp to carry out sterilization treatment. Accordingly, an appropriate cooling device or refrigerating device is preferably used to cool the liquid ML or the plasma-treated solution MLp at 10° C. or lower and to keep the temperature.

Subsequent experiments showed that the half-life of active species depend on a pH value of the plasma-treated solution MLp. To be specific, the experiment was made with a temperature of the plasma-treated solution MLp fixed at 15° C. The experiment showed that, when the pH value of the plasma-treated solution MLp is 5.7, 5.4, 5.3, 5.2, 4.6, 4.5, and 4.1, the half-life [min.] is 0.21, 0.27, 0.39, 0.55, 1.54, 1.43, and 3.77, respectively.

According to the experimental result, lowering the pH value of the plasma-treated solution MLp extends a life time of active species exponentially. It is, therefore, important to keep the pH value of the plasma-treated solution MLp low in order to prolong a life time of active species and to maintain the sterilization effect of the plasma-treated solution MLp.

According to a graph of the foregoing experimental result, when the pH value of the plasma-treated solution MLp is 4.8, the half-life is approximately 1 minute. In such a case, the plasma-treated solution MLp is preferably applied to an object within 1 minute from the production of the plasma-treated solution MLp. The minimum time necessary from the production of the plasma-treated solution MLp to the application thereof to the object is secured. If the plasma-treated solution MLp is neutral (has a pH value of approximately 7.0), the half-life is extremely short. This probably makes it difficult to move the produced plasma-treated solution MLp to another place for application to an object in that place.

In producing the plasma-treated solution MLp, the pH value thereof is decreased due to nitric acid (HNO3) generated by the plasma PM, sometimes decreased to approximately 2.0 or lower. In light of this, the plasma-treated solution MLp is allowed to have a pH value of approximately 2.0 or lower by nitric acid generated by the plasma PM, and the plasma-treated solution MLp is stored as is. This prolongs the half-life of active species greatly.

As described earlier, it is possible to use an agent to adjust, in advance, the plasma-treated solution MLp to have a pH value of approximately 2.0 or lower.

According to the experiments described with reference to FIGS. 5 and 6, a pH value of the plasma-treated solution MLp is approximately 4.0, and the half-life of the active species therein is almost the same as the experimental result.

As discussed earlier, the plasma-treated solution production device 1 is configured of the vessel YK used for generating the plasma-treated solution MLp, the plasma generation device 12 or 12B, the cooling device 11, and so on.

The plasma-treated solution production device 1 configured in this way is installed in, for example, a treatment room of a hospital. The plasma-treated solution production device 1 generates an appropriate amount of plasma-treated solution MLp and holds the same therein. The dropper HK or the like is used, as necessary, to apply the plasma-treated solution MLp to an object. Alternatively, the plasma-treated solution MLp is applied to an object such as medical equipment by immersing the object in the vessel YK holding the plasma-treated solution MLp therein.

Alternatively, the plasma-treated solution MLp generated by the plasma-treated solution production device 1 is used by pressing a button as necessary, and thereby, to put the plasma-treated solution MLp into another small container. Such a small container is preferably made from a thermal insulating material such as polystyrene foam, and is preferably equipped with a function to keep the temperature of the plasma-treated solution MLp low. In applying the plasma-treated solution MLp of the small container to the affected area KS with the dropper HK, the temperature of the plasma-treated solution MLp may be adjusted to return to a room temperature or so to prevent a person from feeling the coldness of the plasma-treated solution MLp.

Yet alternatively, the plasma-treated solution MLp produced by the plasma-treated solution production device 1 may be supplied to a treatment position with a heat insulating tube, and then, may be warmed to a room temperature or so at the outlet of the insulating tube, and applied to the affected area KS.

[Examples of Formulation for Sterilization Use]

The description goes on to a formulation for sterilization use SS in which the plasma-treated solution MLp generated by the plasma-treated solution production device 1 is integrated with a cold insulation material HR.

The formulation for sterilization use SS is made to have a form suitable to carry (move) the plasma-treated solution MLp to a site where sterilization treatment is applied, and to use the same at the site.

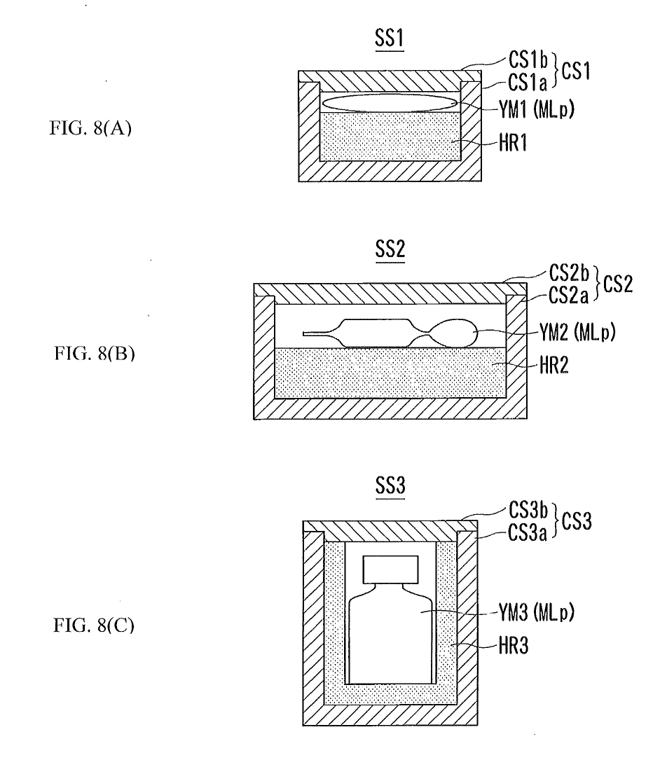

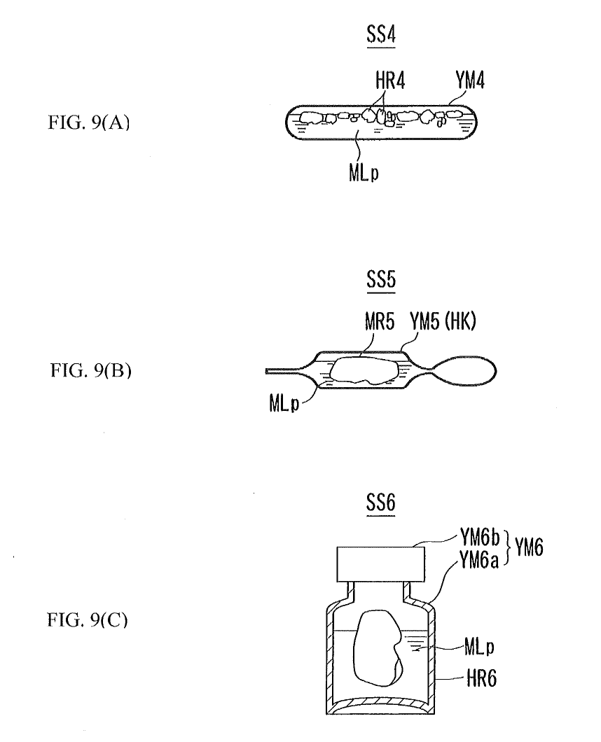

FIGS. 8(A)-8(C) and 9(A)-9(C) show different types of formulations for sterilization use SS.

Referring to FIG. 8(A), a formulation for sterilization use SS1 includes a container CS1 having a container body CS1a and a lid CS1b, a cold insulation material HR1, and a container YM1.

The container YM1 is a pouch member made of a soft synthetic resin or a metal film such as aluminum. The container YM1 contains and seal, therein, a relatively small amount of plasma-treated solution MLp. It is preferable to adjust the plasma-treated solution MLp to have a pH value of 4.8 or lower, and more preferable to adjust the plasma-treated solution MLp to have a pH value of 2.0 or lower.

Examples of the cold insulation material HR1 are a frozen ice gel such as ice-non (registered trademark), an ice bag, ice, dry ice, and a freezing mixture. The cold insulation material HR1 is preferably formed to have an appropriate shape and size to fit in the shape and size of the container YM1 and the container CS1.

The container CS1 contains therein the container YM1 and the cold insulation material HR1. The container CS1 has such a shape and size convenient to carry around. The material of the container CS1 is preferably one having good insulation such as a synthetic resin. The lid CS1b is preferably structured to open and close easily. Accordingly, the lid CS1b may be only inserted in an opening of the container body CS1a. Alternatively, in order to keep the inside temperature of the container CS1 as low as possible, the opening of the container body CS1a may be formed to be sealed with the lid CS1b. Yet alternatively, the opening of the container body CS1a and the lid CS1b may be coupled to each other by screw.

For production of the formulation for sterilization use SS1, the produced plasma-treated solution MLp is held in the container YM1 immediately, and the container YM1 is put in the container body CS1a. The container body CS1a is covered with the lid CS1b to be integrated with each other. The cold insulation material HR1 is prepared in the container body CS1a in advance. Alternatively, the container YM1 is put in the container body CS1a, and after that the cold insulation material HR1 may be put in the container body CS1a.

The formulation for sterilization use SS1 produced may be carried as it is. Instead of this, however, one or more formulations for sterilization use SS1 may be put in an appropriate pouch or box for carriage.

By providing the plasma-treated solution MLp in the form of sterilization use SS1, the temperature of the plasma-treated solution MLp held in the container YM1 is maintained at 10° C. or lower, or 0° C. or so because of the cold insulation material HR1. This prolongs the half-life of the active species, and enables the generated plasma-treated solution MLp to be carried to a distant place where treatment using the plasma-treated solution MLp is applied and to be used in the place. Methods for carrying the formulation for sterilization use SS1 may include using various sorts of package delivery service.

When the formulation for sterilization use SS1 is carried to the place where treatment is applied, the lid CS1b is opened to take out the container YM1, and the plasma-treated solution MLp in the container YM1 is applied to an object for sterilization. For the application of the plasma-treated solution MLp, an appropriate dropper or brush may be used as needed.

Referring to FIG. 8(B), a formulation for sterilization use SS2 includes a container CS2 having a container body CS2a and a lid CS2b, a cold insulation material HR2, and a container YM2.

The container YM2 of the formulation for sterilization use SS2 is a dropper. Accordingly, at a place where treatment is applied, the formulation for sterilization use SS2 is immediately applied to an object using the container YM2 taken out of the container body CS2a to apply sterilization treatment to the object.

Referring to FIG. 8(C), a formulation for sterilization use SS3 includes a container CS3 having a container body CS3a and a lid CS3b, a cold insulation material HR3, and a container YM3.

The container YM3 of the formulation for sterilization use SS3 has a container body YM3a and a lid YM3b. The container YM3 can be carried around with a relatively large amount of the formulation for sterilization use SS3 held therein.

The cold insulation material HR3 has a container-like shape which enables the container YM3 to be held therein. The cold insulation material HR3 has a high cooling effect on the container YM3 because the cold insulation material HR3 covers nearly the entire container YM3.

Referring to FIG. 9(A), a formulation for sterilization use SS4 includes a container YM4 and a cold insulation material HR4. The container YM4 is a pouch member made of a resin film, an aluminum film, or the like. The container YM4 holds the plasma-treated solution MLp therein. The plasma-treated solution MLp has therein the cold insulation material HR4. The cold insulation material HR4 is ice. The plasma-treated solution MLp is cooled by the ice so that the temperature of the plasma-treated solution MLp is kept at 0° C. or so.

Referring to FIG. 9(B), a formulation for sterilization use SS5 includes a container YM5 and a cold insulation material HR5. The container YM5 is a dropper HK. The container YM5 holds the plasma-treated solution MLp therein. The plasma-treated solution MLp has therein ice that is the cold insulation material HR5. The plasma-treated solution MLp is cooled by the cold insulation material HR5, i.e., the ice. The temperature of the plasma-treated solution MLp is kept at 0° C. or so.

The formulation for sterilization use SS5 may be provided by, for example, drawing an appropriate amount of water into the dropper HK as the container YM5, freezing the container YM5 to change the water to ice, and then, drawing the plasma-treated solution MLp into the dropper HK.

The formulation for sterilization use SS5 has a small size. Accordingly, many formulations for sterilization use SS5 can be easily carried by putting the same in an appropriate case. Additionally, the formulations for sterilization use SS5 is used as-is at a site where treatment is applied, so that sterilization treatment can be applied to an object.

Referring to FIG. 9(C), a formulation for sterilization use SS6 includes a container YM6 having a container body YM6a and a lid YM6b, and a cold insulation material HR6. The container. YM6 holds the plasma-treated solution MLp therein. The plasma-treated solution MLp has therein the cold insulation material HR6. The cold insulation material HR6 is ice. The plasma-treated solution MLp is cooled by the ice so that the temperature of the plasma-treated solution MLp is kept at 0° C. or so.

According to the foregoing formulations for sterilization use SS1-SS6, the plasma-treated solution MLp is kept at a low temperature so that biocidal activity can be held for long hours. Therefore, sterilization using plasma can be performed by carrying the plasma-treated solution MLp to a site where no plasma generation device is installed for sterilization treatment and applying the plasma-treated solution MLp to an object.

In the formulation for sterilization use SS1, the container CS1 may double as the cold insulation material HR1 by using a cold insulation material to form the container CS1. This also applies to the formulation for sterilization use SS3. To be specific, the container CS3 may double as the cold insulation material HR3 by using a cold insulation material to form the container CS3. It is also possible to use the structures or materials of the formulations for sterilization use SS1-SS6 in combination.

The types, materials, shapes, and sizes of the container CS, the container YM, and the cold insulation material HR are not limited to the foregoing, and can be arbitrarily modified in various ways.

[Second Embodiment of Sterilization Treatment Method]

The description goes on to an embodiment of a sterilization treatment method with which the plasma-treated solution MLp is frozen to solid ice MS and stored, and thereafter the solid ice MS is thawed for use in sterilization treatment.

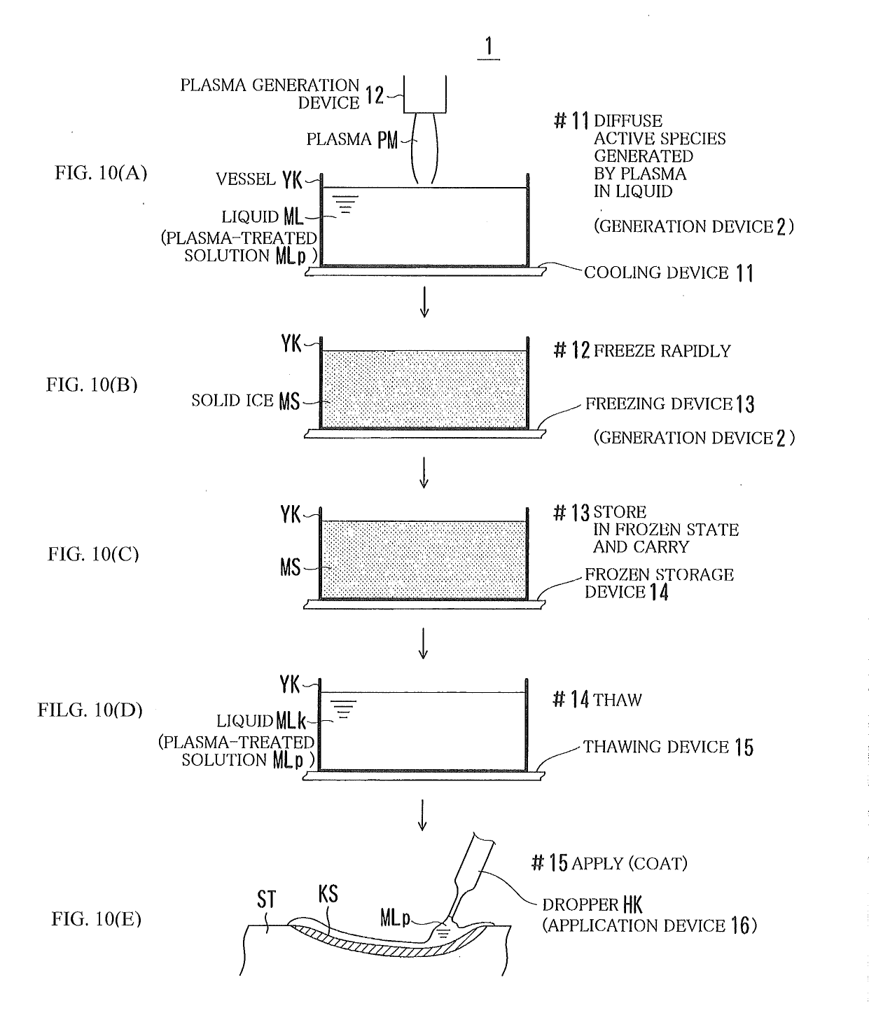

FIG. 10 shows a second example of the sterilization treatment method using the plasma-treated solution production device 1.

As shown in FIG. 10(A), the plasma-treated solution production device 1 is used to produce the plasma-treated solution MLp (Step 411). The devices, operation, and so on of FIG. 10 (A) are the same as those described earlier with reference to FIG. 1(A).

In the second embodiment, however, a pH value of the plasma-treated solution MLp may be adjusted to become 4.8 or lower at a time when the plasma-treated solution MLp which has been frozen to solid ice MS is thawed to return to the liquid MLk, which is described later.

As shown in FIG. 10(B), the plasma-treated solution MLp is quickly frozen (Step 412).

As described earlier, biocidal activity can be held for long hours by lowering the temperature of the plasma-treated solution MLp. The biocidal activity can be held for longer hours by freezing the plasma-treated solution MLp to solid ice.

To be specific, as discussed earlier, when the temperature is 10° C. or so, the lifetime of active species generated in the plasma-treated solution MLp are inactivated in the order of 10 minutes, e.g., approximately 10-20 minutes.

It has been known that the biocidal activity of the plasma-treated solution MLp is attributed not to active species having an extremely short lifetime in the order of microseconds, e.g., OH radicals, but to active species having a lifetime in the order of seconds, e.g., superoxide anion radicals, and to the derivative of the active species. As long as the active species have such a lifetime, the liquid can be frozen through rapid-freezing.

Such rapid-freezing is performed at a temperature of −18° C. or lower, for example. Preferably, the rapid-freezing is performed at a temperature of −30° C. or lower. The rapid-freezing is performed to freeze the plasma-treated solution MLp to solid ice MS. In other words, the solid ice MS is a frozen plasma-treated solution MLp. The temperature of the solid ice MS is set at, for example, −18° C. or lower, or −30° C. or lower.

It is known that the lifetime (half-life) of biocidal activity species in the plasma-treated solution is approximately 2 minutes at a temperature of 20° C. In light of this, for rapid-freezing, if the plasma-treated solution MLp can be frozen for approximately 2 minutes to reach a target temperature of −30° C., biocidal activity loss can be kept at 50% or lower. If the plasma-treated solution MLp can be frozen for twenty seconds or shorter to reach a target temperature of −30° C., 90% of the biocidal activity or more can be held. In contrast, however, if it takes 10 minutes to freeze the plasma-treated solution MLp, the biocidal activity is reduced to approximately one sixteenth of the original biocidal activity. Therefore, it is preferable to quick-freeze the plasma-treated solution MLp in the shortest possible time to reach the target temperature of −30° C., for example.

In the rapid-freezing of this embodiment, the plasma-treated solution MLp is frozen rapidly in a time within which the biocidal activity can be held practically. As shown in FIG. 6, the half-life of active species depends on temperature. Even in a room temperature of 20-25° C., it is not preferable to freeze the plasma-treated solution MLp for hours longer than the half-life of active species. In such a case, the freezing time is preferably 3 minutes or so. In view of this, the biocidal activity can be held by rapidly freezing the plasma-treated solution MLp in which active species are diffused within 3 minutes to change to solid ice MS.

For the rapid-freezing, the freezing device 13, a rapid-freezing device, or the like is used. A freezing device using liquid nitrogen may be also used.

According to the foregoing description, the rapid-freezing is performed after the plasma-treated solution MLp is produced. In such a case, the following arrangement is possible: The liquid ML is adjusted to have a temperature of 0° C. or low so as to be supercooled but not frozen. Immediately after plasma treatment is completed, a stimulus is applied to freeze the plasma-treated solution MLp at once.

Alternatively, a metal container cooled sufficiently is prepared, for example. Then, a small amount of the plasma-treated solution MLp may be put into the metal container to be frozen almost instantaneously.

Yet alternatively, the liquid ML may be dropped in the form of droplets. The droplets may pass through the plasma PM, and a cooled metal plate or the like receives the fallen droplets which are frozen at one time.

Generating the plasma-treated solution MLp may be performed in parallel with freezing the same. For example, while the liquid ML is exposed to the plasma PM, the liquid ML may be cooled to have a lower temperature, and the liquid ML may be frozen gradually from a part thereof which is exposed to the plasma PM for a longer time.

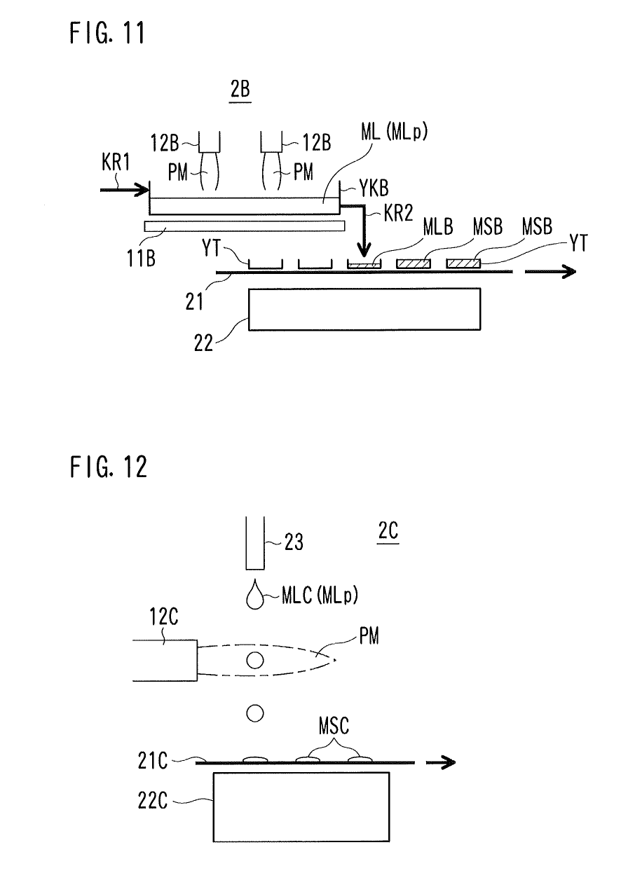

Examples of a device for making the solid ice MS are described later with reference to FIGS. 11-13.

As shown in FIG. 10(C), the solid ice MS is stored in a frozen state (solid ice state) (Step #13). For example, the solid ice MS is stored at a temperature of approximately −18° C. or lower, preferably at a temperature of approximately −30° C. or lower. For the freezing and storing, a frozen storage device 14 is used. The frozen storage device 14 using liquid nitrogen may be used. The frozen storage device 14 may be used also as the freezing device 13, and the solid ice MS may be stored in the freezing device 13 by which the rapid-freezing was performed.

The lower the temperature for freezing and storage is, the less chemical reactions (for example, dismutation reaction) occur in the solid ice MS, so that the biocidal activity by the active species or the derivative thereof are held for long hours. The time of storage changes depending on the storage temperature, for example, for a few hours, one day, a few days, or a few weeks.

The solid ice MS which has been frozen and stored can be carried (or transported) to another location as is. For example, the solid ice MS is carried from a location where the solid ice MS was manufactured to a clinical site where the solid ice MS is required for sterilization or disinfection. Specifically, for example, the solid ice MS is manufactured in large quantities in a factory, and the manufactured solid ice MS is carried to hospitals or research facilities where sterilization and disinfection using the solid ice MS are performed. For the carriage, a freezer car or refrigerator car is used. It is also possible to carry the solid ice MS by appropriate vehicle or aircraft with the solid ice MS frozen and stored using a freezer or liquid nitrogen. At a clinical site, the solid ice MS thus carried is stored in a freezer or the like until it is used.

As shown in FIG. 10 (D), in order to use the solid ice MS for sterilization at the clinical site, a thawing device 15 is used to thaw the solid ice MS (Step #14). The solid ice MS turns to the plasma-treated solution MLp which is the liquid MLk. The superoxide anion radicals and the derivative thereof exist in the plasma-treated solution MLp, and the biocidal activities are held therein.

Thawing the solid ice MS should be performed in the shortest possible time. This is because, after the solid ice MS is thawed to turn to the liquid MLk, if the temperature of the liquid MLk increases, the attenuation speed of the biocidal activity is drastically increased. For example, it is preferable to thaw the solid ice MS at a temperature of 30° C. or so within 1 minute. Heating the solid ice MS with an oven or stove, and heating the solid ice MS at a room temperature or higher are not preferable.

As a heating member used for the thawing, a block of metal having high specific heat and high thermal conductivity such as aluminum is preferably used. The block is set to have an appropriate temperature of approximate 30° C., for example. The solid ice MS is thawed by bringing the same into contact with the block. The volume of the solid ice MS and the volume of the block are so set that the solid ice MS is thawed in the shortest possible time (within 1 minute).

Alternatively, a metal glass syringe of which the temperature is adjustable by blocking heat can be used. The solid ice MS is put into the glass syringe, or, alternatively, the solid ice MS is frozen in the glass syringe. Then, the solid ice MS is gradually ejected from the glass syringe while being thawed therein. In such a case, the thawing of the solid ice MS can be performed simultaneously and in parallel with the application of the plasma-treated solution MLp obtained through the thawing.

Yet alternatively, the solid ice MS may be thawed by heat of an affected area to which the solid ice MS is applied. In such a case, the thawing device 15 is unnecessary. The plasma-treated solution MLp obtained through the thawing by the heat of the affected area and the atmosphere is applied, as is, to the affected area. In order to relieve the coldness of the solid ice MS, for example, a gauze is put onto the affected area to be interposed between the affected area and the solid ice MS.

In the case where the thawing of the solid ice MS and the application thereof are performed in a row, it is preferable to adjust the pH value of the liquid ML that has not yet been frozen to become 4.8 or lower, and to lower the pH value of the solid ice MS from the beginning. In essence, it is desirable to freeze the plasma-treated solution that has been adjusted to have a pH value of 4.8 or lower to produce solid ice.

In the case where a pH value of the plasma-treated solution MLp before being turned to solid ice is not adjusted, it is preferable to adjust the pH value to become 4.8 or lower for example at a time when the solid ice is thawed and turns to the liquid MLk. In such a case, a pH adjustment device having an appropriate structure is used.

As shown in FIG. 10(E), the plasma-treated solution MLp thus thawed is applied to the affected area KS of the living body ST, for example (Step #15). In such a case, the plasma-treated solution MLp is applied to the affected area KS, for example, by dropping the same from the bulb of the dropper HK.

In this way, the affected area KS is sterilized and disinfected. In such a case, the dropper HK is used to draw the plasma-treated solution MLp thereinto and to drop the plasma-treated solution MLp. Alternatively, as described earlier, the solid ice MS is put in the dropper HK. Then, while thawing the solid ice MS, the dropper may drop the plasma-treated solution MLp.

In such a case, for example, the dropper HK is used to draw a predetermined amount of the produced plasma-treated solution MLp thereinto. After that, the freezing device 13 or the like is used to cool the dropper HK in such a manner that the plasma-treated solution MLp in the dropper HK is frozen to the solid ice MS.

In dropping the plasma-treated solution MLp from the dropper HK, the affected area KS may be washed with an acid solution before the plasma-treated solution MLp is applied because bodily fluids of the living body ST sometimes have buffer capacity to keep pH neutral.

When the plasma-treated solution MLp is used, for example, to eradicate pylori bacteria, the plasma-treated solution MLp that has not been subjected to pH adjustment may be applied because a stomach of the living body ST has a low pH value. In such a case, the plasma-treated solution MLp may be applied, for example, by drinking the plasma-treated solution MLp obtained by thawing the solid ice MS, by taking the solid ice MS while being thawed in mouth, by swallowing the solid ice MS as-is, or by using an endoscope to apply the plasma-treated solution MLp. In short, the plasma-treated solution MLp or the solid ice MS can be used exclusively for drinking.

As described earlier, the plasma-treated solution MLp containing active species therein by plasma PM keep the biocidal activity only for a few minutes. The plasma-treated solution MLp, therefore, cannot be delivered to a distant clinical site. However, the plasma-treated solution MLp is frozen to solid ice MS; thereby chemical reactions in the plasma-treated solution MLp occur slowly. This enables the biocidal activity to be held for a long period of time. Therefore, the plasma-treated solution MLp in the form of solid ice MS is delivered to many places, so that the plasma-treated solution MLp can be used as a liquid for sterilization in a clinical site where no plasma generation device 12 is installed.

According to this embodiment, sterilization using the plasma PM can be performed in a place where no plasma generation device 12 is installed. Specifically, if a freezer is installed in a clinical site, the solid ice MS can be frozen and stored. When necessary, the solid ice MS may be thawed for use as an antiseptic or a therapeutic agent.

The plasma-treated solution MLp may be used, for example, as an antiseptic or a therapeutic agent for odontotherapy, or a surgical antiseptic for bedsore or the like. To be specific, the plasma-treated solution MLp may be used, for example, for root canal sterilization or periodontal disease in dental treatment. The plasma-treated solution MLp may be used also for surgical treatment, e.g., treatment for intractable infection occurring in site of trauma such as burn or bedsore, diabetes, or gangrene prevention due to frostbite.

According to this embodiment, the application of the plasma-treated solution MLp in odontotherapy or surgical treatment is used safer and easier as compared with the case of direct emission of plasma PM and emission of afterglow gas.

In the description with reference to FIGS. 10(A)-10(E), the generation device 2 for solid ice for sterilization use is configured of the cooling device 11, the plasma generation device 12, and the freezing device 13 used in FIGS. 10(A) and 10(B), and is further configured of the pH adjustment device or the frozen storage device 14.

[Examples of Device for Producing Solid Ice for Sterilization Use]

The description goes on to examples of a device for producing solid ice for sterilization use with reference to FIGS. 11-13.

Referring to FIG. 11, a production device 2B includes a cooling device 11B, a plurality of plasma generation devices 12B, a vessel YKB, frozen vessels YT, a conveyor 21, and a freezing device 22.

The vessel YKB is provided with an inlet pipe line KR1 and an outlet pipe line KR2 for a liquid ML. The vessel YKB contains the liquid ML therein, and the liquid ML corresponding to the amount of the liquid ML flowing from the outlet pipe line KR2 is refilled from the inlet pipe line KR1. The liquid ML in the vessel YKB flows slowly from the inlet pipe line KR1 side to the outlet pipe line KR2 side. The liquid ML that has reached the vicinity of the outlet pipe line KR2 already changes to the plasma-treated solution MLp. The vessel YKB is cooled by the cooling device 11, so that the temperature of the liquid ML and the plasma-treated solution MLp is set at 10° C. or lower.

The plasma generation devices 12B are placed above the liquid ML of the vessel YKB in such a manner that the plasma PM generated from the plasma generation devices 12B is emitted to the liquid ML. Since a plurality of the plasma generation device 12B is provided, the liquid ML flowing in the vessel YKB is sufficiently exposed to the plasma PM, contacts active species generated by the plasma PM sufficiently, and thereby, the active species are diffused in the plasma-treated solution MLp.

The frozen vessel YT is made of a metal having high specific heat and high thermal conductivity such as aluminum. The frozen vessels YT are carried forward in order by the conveyor 21. The conveyor 21 stops at a time when the frozen vessel YT arrives at a position directly below the outlet pipe line KR2, and moves again after a predetermined amount of the plasma-treated solution MLp is supplied from the outlet pipe line KR2 to the frozen vessel YT directly below the outlet pipe line KR2. In this example, the conveyor 21 and the outlet pipe line KR2 are one example of a device for containing the plasma-treated solution MLp in the vessel. The frozen vessels YT are sufficiently cooled to, for example, approximately −30° C. by the freezing device 22, which enables the liquid ML dropping in the frozen vessel YT to be frozen almost instantaneously.

The plasma-treated solution MLp in the vessel YKB drops, by each predetermined amount thereof, from the outlet pipe line KR2 toward the frozen vessel YT where the dropped plasma-treated solution MLp is frozen rapidly (instantaneously) to solid ice MSB.

The solid ice MSB in the frozen vessel YT is carried by the conveyor 21 to an appropriate post-processing device. In the post-processing device, for example, the solid ice MSB is taken out of the frozen vessel YT and is frozen/stored. In such a case, a plurality of the frozen vessels YT may be coupled to one another. Accordingly, it is also possible to form the frozen vessel YT by the entirety of the conveyor 21.

Alternatively, the solid ice MSB can be frozen and stored as-is without being taken out of the frozen vessel YT. In such a case, the frozen vessel YT may be made of a thin metal film such as an aluminum foil or aluminum wrap to wrap around the solid ice MS inside the frozen vessel YT.

As described above, the production device 2B is used to supply the plasma-treated solution MLp by a predetermined flow rate from the vessel YKB, and the plasma-treated solution MLp is fed to the frozen vessel YT to be frozen quickly. This enables continuous production of easy-to-use solid ice MS having an appropriate size (volume).

In the illustrated example of FIG. 11, the plasma generation devices 12B are so placed that the plasma PM is jetted out vertically. Instead of this, however, each of the plasma generation devices 12B may be so placed that the plasma PM is jetted out horizontally in parallel with the surface of the liquid ML. Another configuration is possible in which the vessel YKB itself functions as a flow passage for the liquid ML.

In the example of FIG. 11, a plurality of the plasma generation devices 12B is used. Instead of this, a plasma generation device may be used which applies plasma treatment successively to the liquid ML flowing in the vessel YKB. For example, it is possible to use a plasma generation device in which a high-voltage electrode and the ground electrode are disposed above and below the vessel YKB to sandwich the vessel YKB therebetween, and a high voltage is applied across the electrodes to perform dielectric barrier discharge.

Referring to FIG. 12, a production device 2C includes a plasma generation device 12C, a conveyor 21C, a freezing device 22C, and a liquid supplying device 23.

The liquid supplying device 23 drops an appropriate amount of liquid MLC continuously in a manner to pass the liquid MLC through the plasma PM generated by the plasma generation device 12C. The liquid MLC passes through the plasma PM and the vicinity thereof, so that active species are diffused in the liquid MLC which turns to the plasma-treated solution MLp.

The conveyor 21C includes a belt made of a material having good thermal conductivity such as metal. The conveyor 21C is cooled to a temperature of approximately −30° C. or lower by the freezing device 22C. The conveyor 21C is so placed to run below the liquid supplying device 23. Alternatively, a freezing conveyor device may be used in which the freezing device 22C and the conveyor 21C are integrated with each other.

The liquid MLC which drops from the liquid supplying device 23 to turn to the plasma-treated solution MLp falls on the conveyor 21C. Each drop of the plasma-treated solution MLp is frozen rapidly to solid ice MSC. The solid ice MSC is carried forward, as-is, by the conveyor 21C. The solid ice MSC delivered by the conveyor 21C is put into an appropriate container, or, wrapped in an appropriate package, and is stored in a frozen storage device, or delivered to another location.

As described above, the production device 2C continuously produces the solid ice MSB for sterilization use. The size, volume, or shape of the solid ice MSC is preferably determined depending on an object for which the solid ice MSC is used.

In the illustrated example of FIG. 12, the plasma generation device 12C is so placed that the plasma PM is jetted out horizontally. Instead of this arrangement, the plasma generation device 12C may be so placed that the plasma PM is jetted out vertically. In such a case, the liquid supplying device 23 is preferably placed in such a manner that the liquid MLC drops through the plasma PM jetted out vertically. This arrangement increases the time during which the liquid MLC contacts the plasma PM before dropping off, thereby active species are diffused in the liquid MLC with reliability.

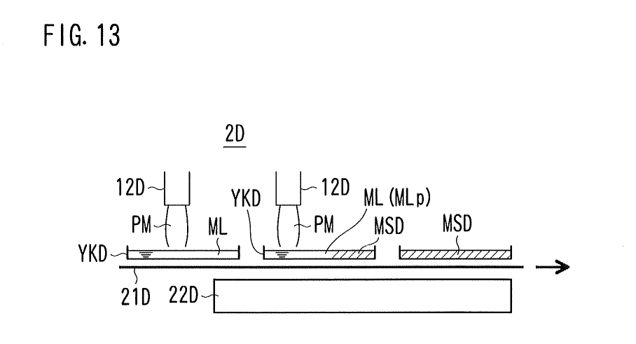

Referring to FIG. 13, a production device 12D includes a plurality of plasma generation devices 12D, vessels YKD, a conveyor 21D, and a freezing device 22D.

The vessels YKD are made of metal. The vessels YKD are placed on the conveyor 21D and carried forward in order at a low speed. As being carried forward, the vessels YKD are cooled to a low temperature by the freezing device 22D.

The plasma generation devices 12D are placed above the conveyor 21D to emit plasma PM onto the surface of the liquid ML in the vessel YKD.

The liquid ML is cooled to a temperature of 10° C. or lower and is exposed to the plasma PM, so that active species are diffused in the liquid ML. As being carried by the conveyor 21D, the liquid ML is gradually cooled. The liquid ML starts to be frozen from a part thereof that has been exposed to the plasma PM, and turns to the solid ice MSD.

In this way, according to the production device 2D, producing the plasma-treated solution MLp and freezing the same are performed in parallel with each other to overlap temporally. Consequently, the solid ice MOD for sterilization use is produced continuously.

Another arrangement is possible in which the liquid ML in the vessels YKD is frozen (iced) from the beginning, and the ice (iced liquid ML) in the vessels YKD is exposed to the plasma PM from the plasma generation devices 12D. In such a case, the ice in the vessels YKD is exposed to the plasma PM, and the ice melts gradually from the surface thereof with the heat of the plasma PM, so that the ice returns to the liquid ML. Active species are diffused in the liquid ML, which produces the plasma-treated solution MLp. The plasma-treated solution MLp produced is cooled by the freezing device 22D and is frozen again to the solid ice MSD. The positions of the vessels YKD, the plasma generation devices 12D, and the freezing device 22D, the depth and length of the vessel YKD, and the speed of the conveyor 21D are preferably adjusted in such a manner that melting of the iced liquid ML, plasma treatment on the liquid ML, and re-freezing of the plasma-treated solution MLp are performed smoothly in order.

This makes it possible to perform all the steps from production of the plasma-treated solution MLp to production of the solid ice MS in a temperature around 0 degrees. This enables effective production of the solid ice MS having a high density of active species.

[Examples of Solid Ice for Sterilization Use]

The description goes on to various types of solid ice MS obtained by freezing the plasma-treated solution MLp. To be specific, the description provides an example of a suitable style in which the solid ice MS is carried (or moved) to a place where sterilization treatment is performed and is used therein, although the method for producing the solid ice MS itself is detailed earlier.

The following description takes examples of solid ice for sterilization use ST containing various types of solid ice MS. It can be said that the solid ice for sterilization use ST described herein is another embodiment of a “formulation for sterilization use” using the plasma-treated solution MLp. The solid ice for sterilization use ST uses solid ice MS obtained by freezing the plasma-treated solution MLp; therefore the solid ice MS itself serves as a cold insulation material.

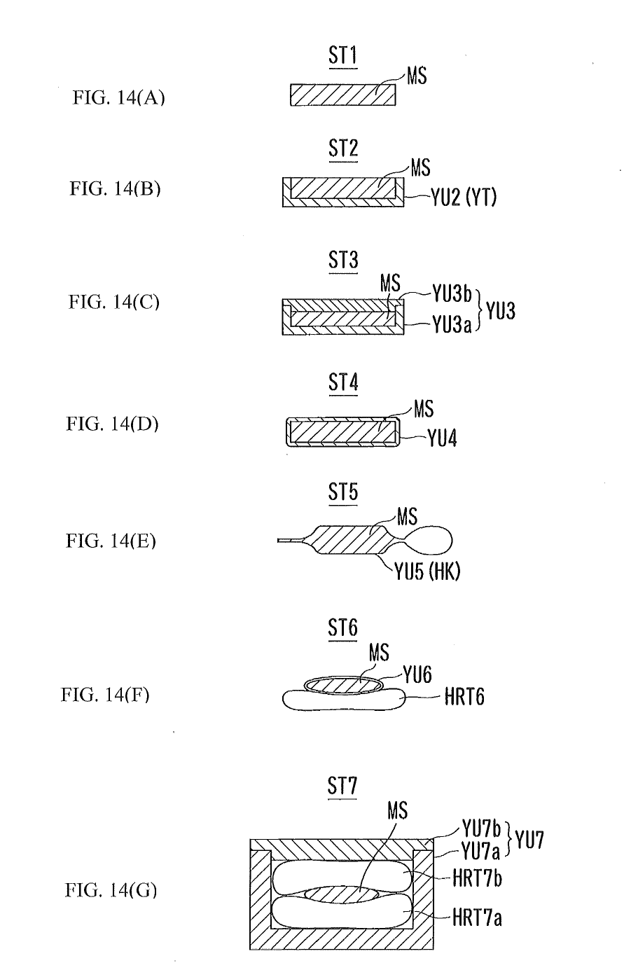

FIGS. 14(A)-14(G) show various types of solid ice for sterilization use ST.

In FIG. 14(A), solid ice for sterilization use ST1 is solid ice MS itself. The solid ice MS may have any shape such as a plate, a rectangular parallelepiped, a sphere, a disc, or a bar. The solid ice MS may have any size of a small size for single use only, a relatively large size for twice use or more, or a large size which enables division of the solid ice MS into a plurality sets of solid ice MS.

In FIG. 14(B), solid ice for sterilization use ST2 is provided in the form of solid ice MS held in a container YU2. The container YU2 may have any shape and any size depending on the shape and size of the solid ice MS. The container YU may be made of, for example, a metal such as steel or aluminum or a synthetic resin. The container YU2 may be the frozen vessel YT used in the production device 2B as discussed earlier.

In FIG. 14(C), solid ice for sterilization use ST3 is provided in the form of solid ice MS held in a container YU3. The container YU3 is provided with a container body YU3a and a lid YU3b to house the solid ice MS therein. The temperature of the solid ice MS can be maintained at a low temperature for long hours by using, as the material of the container YU3, one having good thermal insulation.

In FIG. 14(D), solid ice for sterilization use ST4 is provided in the form of solid ice MS of which the entire outer surface is covered with a container YU4 made of a wrapping material. The container YU4 may be a metal film such as an aluminum foil.

In FIG. 14(E), solid ice for sterilization use ST5 is provided in the form of solid ice MS held in a container YU5 as a dropper. The various droppers HK described earlier may be used as the container YU5. The dropper HK draws an unfrozen plasma-treated solution MLp thereinto and the resultant is frozen, so that the solid ice for sterilization use ST5 is produced. The solid ice MS is melted down partly, and the plasma-treated solution MLp obtained by melting the solid ice MS can be directly applied to an object with the dropper HK serving also as the container YU5.

In FIG. 14(F), solid ice for sterilization use ST6 is provided in the form of solid ice MS covered with a container YU6 made of a wrapping material, and the resultant is brought into contact with the cold insulation material HRT6 and is integrated therewith. The resultant may be wrapped with an appropriate wrapping material. As the cold insulation material HRT6, the various cold insulation materials HR discussed above may be used. In particular, the solid ice MS is usually frozen in 0° C. or lower, the cold insulation material HRT is preferably dry ice or a freezing mixture such as an organic solvent.

In FIG. 14(G), solid ice for sterilization use ST7 includes a container YU7 having a container body YU7a and a lid YU7b, cold insulation materials HRT7a and HRT7b, and solid ice MS. The container YU7 may be the one similar to the container CS1 shown in FIG. 8(A). The solid ice MS may be wrapped around with an appropriate wrapping material.

According to the solid ice for sterilization use ST1-ST7, the solid ice MS contained therein is thawed to turn to the plasma-treated solution MLp (treated solution MLk). This enables sterilization using plasma to be performed at a site where no plasma generation device is installed. In addition, the solid ice MS is frozen and the temperature thereof is usually maintained at 0° C. or lower. Therefore, the biocidal activity is maintained for long hours.

The structures or materials of the solid ice for sterilization use ST1-ST7 may be combined with one another.

Further, the type, material, shape, or size of the container YU and the cold insulation material HRT are not limited to those discussed above and may be changed in different ways.

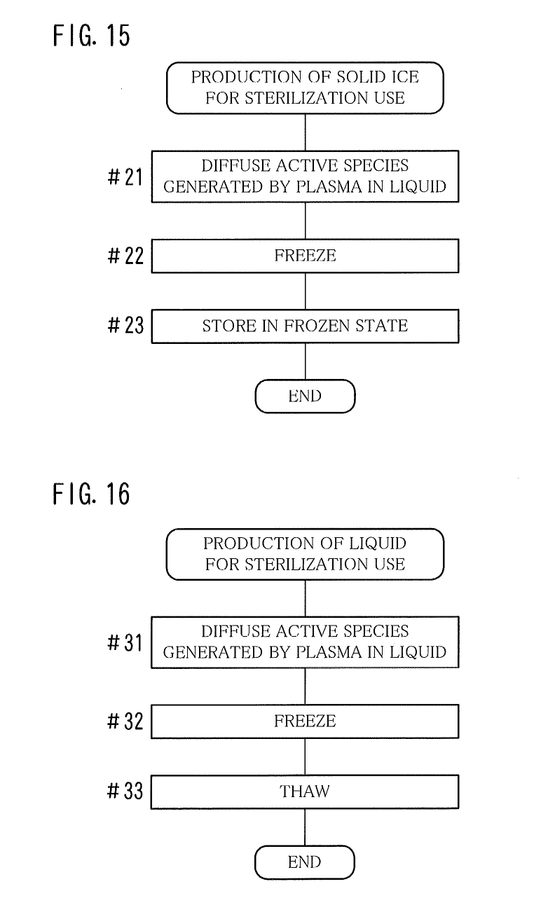

FIG. 15 depicts a method for producing solid ice for sterilization use. According to the method, active species generated by plasma PM are brought into contact with a liquid ML, and are diffused in the liquid ML (Step #21). The liquid ML in which the active species are diffused is then frozen to solid ice MS (Step #22). The solid ice MS is frozen and stored if necessary (Step #23). The frozen and stored solid ice MS can be carried to a distance place.

FIG. 16 depicts a method for producing a liquid for sterilization use. According to the method, active species generated by plasma PM are brought into contact with a liquid ML, and are diffused in the liquid ML (Step #31). The liquid ML in which the active species are diffused is then frozen to solid ice MS (Step #32). The solid ice MS is thawed to turn to liquid MLk (Step #33).

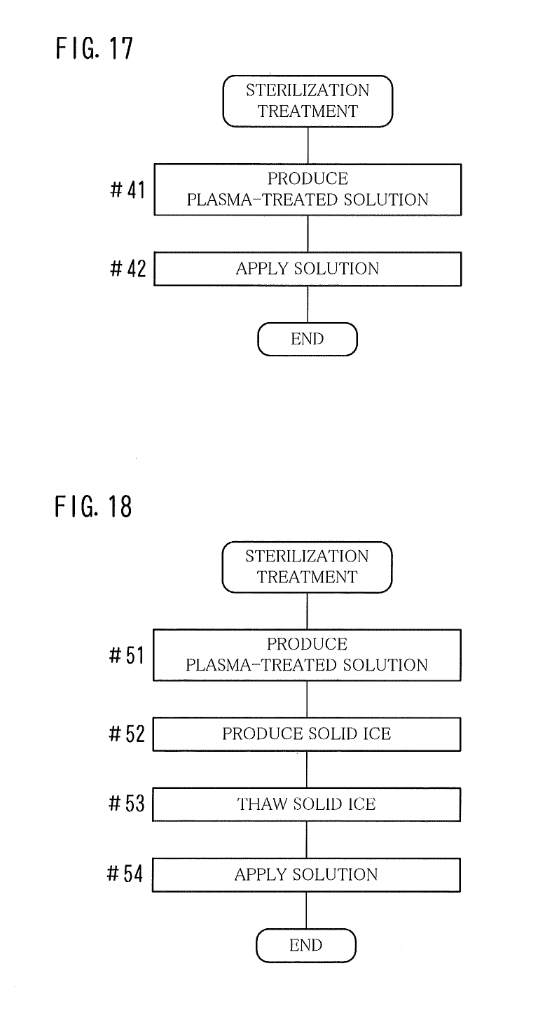

FIG. 17 depicts a sterilization treatment method. According to the method, a plasma-treated solution MLp is produced (Step #41), and is applied to an object for sterilization treatment (Step #42). In Step #41, the temperature of the plasma-treated solution MLp is preferably set at 10° C. or lower.

FIG. 18 depicts a sterilization treatment method. According to the method, a plasma-treated solution MLp is produced (Step #51), and is frozen to produce solid ice MS (Step #52). The solid ice MS is carried, as need arises, to a place where treatment is applied. The solid ice MS is thawed at the place where treatment is applied (Step #53). The liquid MLk obtained by thawing the solid ice MS is applied to an object (Step #54).

In Step #41 or Step #51 where a plasma-treated solution is produced, a liquid which has been adjusted to have a pH value of 4.8 or lower may be used. Alternatively, after the plasma-treated solution MLp is produced in Step #41 or Step #51, a pH value of the plasma-treated solution MLp may be adjusted to become 4.8 or lower. After the solid ice MS is thawed in Step #53, a pH value of the plasma-treated solution MLk may be adjusted to become 4.8 or lower. At any rate, it is preferable to adjust the pH value of the plasma-treated solution MLp or the liquid MLk to become 4.8 or lower before the plasma-treated solution MLp or the liquid MLk is applied to an object.

The description goes on to results of experiments conducted on biocidal activities of the plasma-treated solution MLp and the solid ice MS.

[Experimental Result 1]

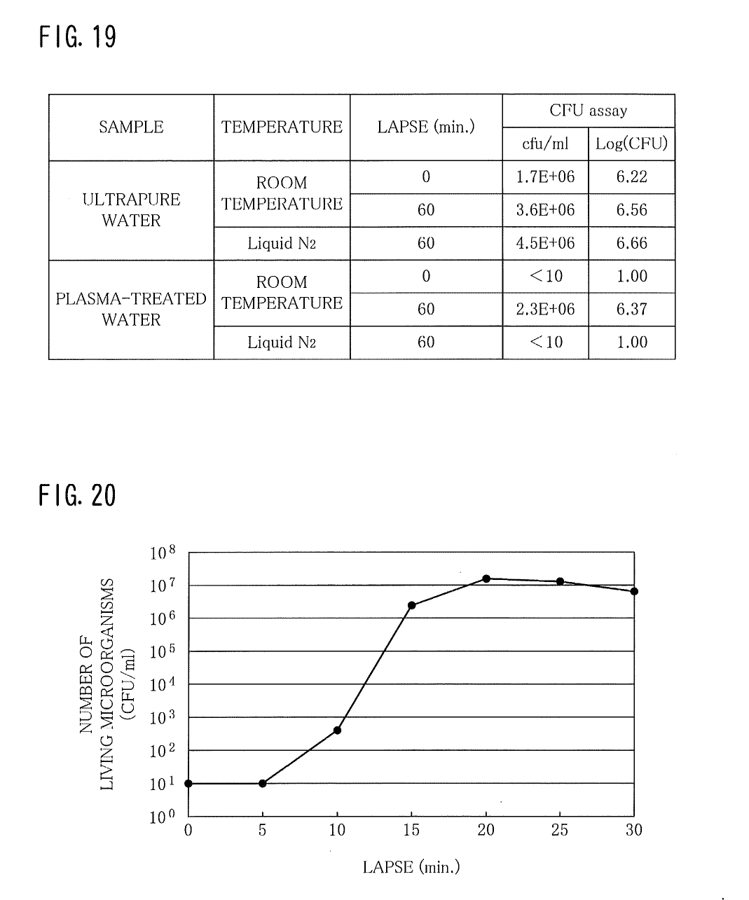

FIG. 19 is a table of experimental results for verifying a sterilization effect in a plasma-treated water.

For the experiment, ultrapure water is prepared. The ultrapure water is divided into plasma-treated water MLCp that has been exposed to plasma PM, and ultrapure water (plasma unexposed water) MLCm that has not been exposed to the plasma PM. Then, the plasma-treated water MLCp is stored at a room temperature, and is left as-is for a predetermined time. After that, the plasma-treated water MLCp is mixed with a set of microbial suspension containing Escherichia coli of which a pH value is adjusted to become 3.7, and evaluation is performed. On the other hand, the plasma-treated water MLCp is frozen and stored by quick-freezing using liquid nitrogen, and is left as-is for a predetermined time. After that, the plasma-treated water MLCp is mixed with a set of microbial suspension containing Escherichia coli of which a pH value is adjusted to become 3.7, and evaluation is performed. Further, both the evaluations are also made for the plasma unexposed water MLCm under the same conditions as that for the plasma-treated water MLCp.

As denoted in FIG. 19, as for the ultrapure water (plasma unexposed water MLCm), the fungicidal concentration is approximately 3×10⁶ CFU/ml after 60 minutes have elapsed for each of the cases where the plasma unexposed water MLCm is stored at a room temperature and where the plasma unexposed water MLCm is frozen and stored. In short, the fungicidal concentration does not change between the storage at the room temperature and the frozen storage.

As for the plasma-treated water MLCp, the Escherichia coli is sterilized down to a limit for detection of 10 CFU/ml or lower immediately after (0 minutes after) plasma treatment is performed. After the plasma-treated water MLCp is left as-is at a room temperature for 60 minutes, none of microbicidal activity is seen. In contrast, in the case where the plasma-treated water MLCp is subjected to the plasma treatment, and is frozen and stored using liquid nitrogen, the Escherichia coli is detected below the limit for detection. In short, as with the case of immediately after the plasma treatment is applied, microbicidal activities are held sufficiently.

The experimental results show that the sterilization using the plasma-treated solution MLp according to the embodiment is different, in mechanism of action, from sterilization using stable chemical species having a half-life longer than 60 minutes, e.g., ozone generated by plasma or hydrogen peroxide.

Incidentally, as the ultrapure water, water having an electric resistance of 18.2MΩ/cm or greater is used.

[Experimental Result 2]

FIG. 20 is a graph showing the residual biocidal activity in a plasma-treated water and a time variation.

For the experiment, ultrapure water is prepared. The ultrapure water is exposed to plasma PM for 5 minutes to produce a plasma-treated water MLDp. The produced plasma-treated water MLDp is mixed with a set of microbial suspension buffer solution at a room temperature. The mixture is left as-is at a room temperature for 5 minutes, serially diluted, applied onto a plate, and measured for the number of colonies.

As denoted in FIG. 20, sterilization can be performed up to the limit for detection immediately after (0 minutes after) plasma treatment is performed. Since no changes are seen in the number of living microorganisms before the lapse time reaches 5 minutes, this shows that microbicidal activities remain in the liquid. Thereafter, the residual biocidal activities decrease with time, and the microbicidal activities are lost almost completely in about 10-20 minutes.

The experimental results show that it is impossible to keep the biocidal activity of the plasma-treated water MLDp at a room temperature.

[Experimental Result 3]

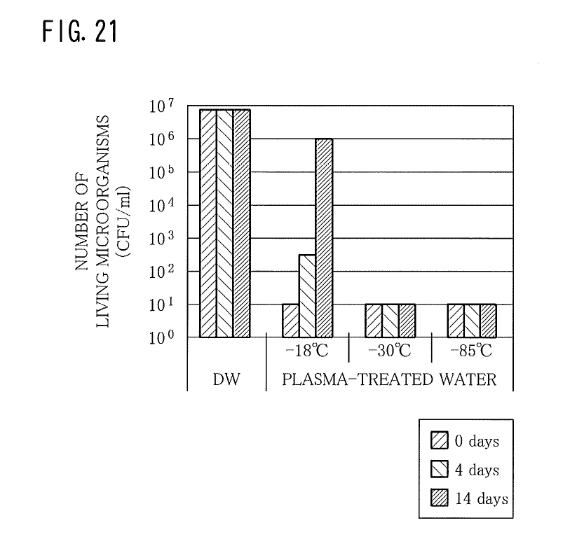

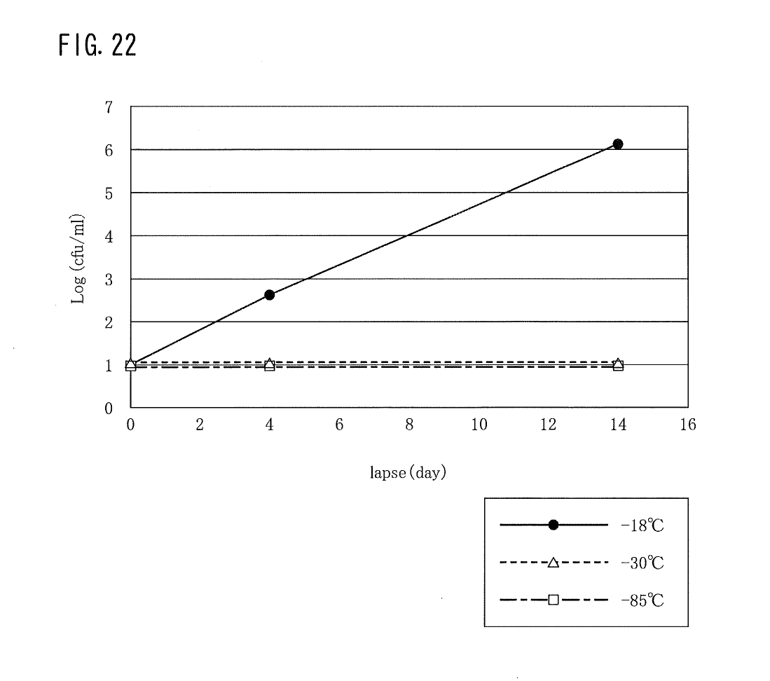

FIG. 21 is a graph showing the relationship between a freezing temperature of a plasma-treated water and a sterilization effect; and FIG. 22 is a graph showing the relationship between a frozen and storage time of a plasma-treated water and a sterilization effect. The graph of FIG. 22 shows, in the vertical axis, a logarithm of the number of living microorganisms indicated in FIG. 21 in the plasma-treated water and, in the horizontal axis, a period of time during which the plasma-treated water is frozen and stored.

For the experiment, a liquid MLE is prepared. The liquid MLE is exposed to plasma PM to produce a plasma-treated water (plasma sterilization water) MLEp. The plasma-treated water MLEp is frozen rapidly to produce solid ice blocks MSE. The solid ice blocks MSE are frozen and stored at a temperature of −18° C., −30° C., and −85° C., respectively. After a predetermined lapse of time, the frozen solid ice blocks MSE are thawed on ice to mix with a set of microbial suspension buffer solution. The mixture is left as-is at a room temperature for 5 minutes, serially diluted, applied onto a plate, and measured for the number of colonies.

According to the graphs of FIGS. 21 and 22, in the case where the plasma-treated water is stored at −18° C., the microbicidal activity decreases gradually with time, and almost no biocidal activity is observed in two weeks.

In contrast, in the case where the plasma-treated water is stored at −30° C. or lower, the biocidal activity still remains high even two weeks later.

As known from the experiment results, it is possible to transport and store the plasma-treated water with the biocidal activity maintained by rapidly freezing the plasma-treated water and storing the same at −30° C. or lower.

In the embodiments discussed above, the configurations of all or part of the vessels YK, YKB, and YKD, the liquid ML, the cooling device 11, the plasma generation devices 12, 12B-12D, the freezing devices 13, 22, 22C, 22D, the frozen storage device 14, the thawing device 15, the application device 16, the liquid supplying device 23, the pH adjustment device, the generation devices 1, 1B-1D, and other elements, shapes, dimensions, quantities, materials, arrangements, components, temperatures, periods, and so on can be arbitrarily modified in various ways within the spirit of the present invention.

The present invention can be applied to sterilization or disinfection of medical equipment, pharmaceutical agents, living bodies, and so on, and also applied to production of formulation for sterilization use and solid ice for sterilization use.

1. A sterilization treatment method comprising:
producing a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid;
freezing the plasma-treated solution to produce solid ice and store the solid ice in a frozen state;
thawing the solid ice to return to the plasma-treated solution in which biocidal activity by the superoxide anion radicals (O₂⁻.) or the precursor of the superoxide anion radicals (O₂⁻.) is held; and
applying a sterilization treatment by any one of the following:
allowing the plasma-treated solution to have a pH value of 4.8 or lower to apply the resultant plasma-treated solution to an object, and
applying the plasma-treated solution to an object of which a pH value is 4.8 or lower.

2. The sterilization treatment method according to claim 1, wherein
the solid ice is thawed to adjust the plasma-treated solution in which biocidal activity by the superoxide anion radicals (O₂⁻.) or the precursor of the superoxide anion radicals (O₂⁻.) is held to have a pH value of 4.8 or lower to increase protons (H⁺) in the plasma-treated solution, and
the resultant plasma-treated solution is applied to the object.

3. A method for producing solid ice for sterilization use, the solid ice being thawed to a plasma-treated solution to be used as a liquid for sterilization, the method comprising:
producing a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid; and
freezing the plasma-treated solution to produce solid ice, the sold ice, when being thawed, returning to the plasma-treated solution in which biocidal activity by the superoxide anion radicals (O₂⁻.) or the precursor of the superoxide anion radicals (O₂⁻.) is held, and using the sold ice produced as the solid ice for sterilization use.

4. The method for producing solid ice for sterilization use according to claim 3, wherein
a temperature of the liquid is kept at 10° C. or lower,
the plasma-treated solution is produced at 10° C. or lower, and
the plasma-treated solution is frozen rapidly to produce the solid ice within 3 minutes.

5. The method for producing solid ice for sterilization use according to claim 3, wherein
the plasma-treated solution is adjusted to have a pH value of 4.8 or lower to increase protons (H⁺) in the plasma-treated solution, and
the resultant plasma-treated solution is frozen to produce the solid ice.

6. Solid ice for sterilization use produced by a method according to claim 3.

7. A device for producing solid ice for sterilization use, the solid ice being thawed to a plasma-treated solution to be used as a liquid for sterilization, the device comprising:
a plasma-treated solution production device configured to produce a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid; and
a freezing device configured to freeze the plasma-treated solution to produce solid ice, the sold ice, when being thawed, returning to the plasma-treated solution in which biocidal activity by the superoxide anion radicals (O₂⁻.) or the precursor of the superoxide anion radicals (O₂⁻.) is held, and to use the sold ice produced as the solid ice for sterilization use.

8. The device for producing solid ice for sterilization use according to claim 7, wherein
the plasma-treated solution production device is provided with a liquid supply device configured to drop the liquid in the vicinity of the plasma or to drop the liquid to pass through the plasma, and
the freezing device receives the liquid dropped by the liquid supply device to freeze the liquid quickly.

9. The device for producing solid ice for sterilization use according to claim 7, wherein
the plasma-treated solution production device generates plasma in the vicinity of the liquid held in a container or in a manner to make contact with the liquid,
the freezing device is so placed as to refrigerate the container, and
production of the plasma-treated solution and freezing of the plasma-treated solution are performed in parallel with each other.

10. The device for producing solid ice for sterilization use according to claim 7, wherein the plasma-treated solution production device is configured to emit plasma to the frozen liquid to melt the frozen liquid to diffuse, in the liquid melted, the superoxide anion radicals (O₂⁻.) or the precursor of the superoxide anion radicals (O₂⁻.) to produce the plasma-treated solution.

11. The device for producing solid ice for sterilization use according to claim 7, comprising a pH adjustment device configured to adjust the plasma-treated solution to have a pH value of 4.8 or lower.

12. A method for producing a liquid for sterilization use, the method comprising: producing a plasma-treated solution in which biocidal activity is held by diffusing, in a liquid, superoxide anion radicals (O₂⁻.) or a precursor of the superoxide anion radicals (O₂⁻.) by plasma generated in a vicinity of or in a manner to make contact with the liquid;
freezing the plasma-treated solution to produce solid ice and store the solid ice in a frozen state; and
thawing the solid ice stored in the frozen state to return to the plasma-treated solution in which biocidal activity by the superoxide anion radicals (O₂⁻.) or the precursor of the superoxide anion radicals (O₂⁻.) is held, and allowing the plasma-treated solution to have a pH value of 4.8 or lower to use the resultant plasma-treated solution as the liquid for sterilization use.

13. The sterilization treatment method according to claim 1, wherein
in producing the plasma-treated solution, the plasma is generated in the vicinity of the liquid held in a container or in a manner to make contact with the liquid,
in producing the solid ice, the container is refrigerated, and
production of the plasma-treated solution and freezing of the plasma-treated solution are performed in parallel with each other.

14. The sterilization treatment method according to claim 1, wherein, in producing the plasma-treated solution, plasma is emitted to the frozen liquid to melt the frozen liquid, and the superoxide anion radicals (O₂⁻.) or the precursor of the superoxide anion radicals (O₂⁻.) are diffused in the liquid melted to produce the plasma-treated solution.

15. The method for producing a liquid for sterilization use according to claim 12, wherein
in producing the plasma-treated solution, the plasma is generated in the vicinity of the liquid held in a container or in a manner to make contact with the liquid,
in producing the solid ice, the container is refrigerated, and
production of the plasma-treated solution and freezing of the plasma-treated solution are performed in parallel with each other.

16. The method for producing a liquid for sterilization use according to claim 12, wherein, in producing the plasma-treated solution, plasma is emitted to the frozen liquid to melt the frozen liquid, and the superoxide anion radicals (O₂⁻.) or the precursor of the superoxide anion radicals (O₂⁻.) are diffused in the liquid melted to produce the plasma-treated solution.

17. Solid ice for sterilization use exclusively for drinking produced by a method according to claim 3.
